SEQUENCE LISTING (13 sequences)

SEQ ID NO: 1 - DNA, 29 bp, synthetic construct
  source 1..29: mol_type = other DNA
  modified_base 23: mod_base = OTHER; note = Uracil
aatgatacggcgaccaccgagatctacac

SEQ ID NO: 2 - DNA, 24 bp, synthetic construct
  source 1..24: mol_type = other DNA
  modified_base 22: mod_base = OTHER; note = 8-oxoguanine
caagcagaagacggcatacganat

SEQ ID NO: 3 - DNA, 24 bp, synthetic construct
  source 1..24: mol_type = other DNA
  modified_base 20: mod_base = OTHER; note = 8-oxoguanine
caagcagaagacggcatacnagat

SEQ ID NO: 4 - DNA, 24 bp, synthetic construct
  source 1..24: mol_type = other DNA
  modified_base 20: mod_base = OTHER; note = 8-oxoguanine
  modified_base 22: mod_base = OTHER; note = 8-oxoguanine
caagcagaagacggcatacnanat

SEQ ID NO: 5 - DNA, 29 bp, synthetic construct
  source 1..29: mol_type = other DNA
  misc_difference 23: note = Allyl-T
aatgatacggcgaccaccgaganctacac

SEQ ID NO: 6 - DNA, 35 bp, synthetic construct
  source 1..35: mol_type = other DNA
gctggcacgtccgaacgcttcgttaatccgttgag

SEQ ID NO: 7 - DNA, 35 bp, synthetic construct
  source 1..35: mol_type = other DNA
ctcaacggattaacgaagcgttcggacgtgccagc

SEQ ID NO: 8 - DNA, 35 bp, synthetic construct
  source 1..35: mol_type = other DNA
cgtcgtctgccatggcgcttcggtggatatgaact

SEQ ID NO: 9 - DNA, 35 bp, synthetic construct
  source 1..35: mol_type = other DNA
agttcatatccaccgaagcgccatggcagacgacg

SEQ ID NO: 10 - DNA, 35 bp, synthetic construct
  source 1..35: mol_type = other DNA
acggccgctaatatcaacgcgtcgaatccgcaact

SEQ ID NO: 11 - DNA, 35 bp, synthetic construct
  source 1..35: mol_type = other DNA
agttgcggattcgacgcgttgatattagcggccgt

SEQ ID NO: 12 - DNA, 34 bp, synthetic construct
  source 1..34: mol_type = other DNA
gccgcgttacgttagccggactattcgatgcagc

SEQ ID NO: 13 - DNA, 34 bp, synthetic construct
  source 1..34: mol_type = other DNA
gctgcatcgaatagtccggctaacgtaacgcggc

(19) United States
(12) Patent Application Publication
(10) Pub. No.: US 2024/0002933 A1
(43) Pub. Date: Jan. 4, 2024

(54) SEQUENCING NANOPARTICLES AND METHODS OF MAKING THE SAME

(71) Applicant: ILLUMINA CAMBRIDGE LIMITED, Cambridge (GB)

(72) Inventors: Amani El Fagui, Slough (GB); Wayne N. George, Ilford (GB)

(21) Appl. No.: 18/343,696

(22) Filed: Jun. 28, 2023

Related U.S. Application Data
(60) Provisional application No. 63/357,479, filed on Jun. 30, 2022.

(51) Int. Cl. C12Q 1/6874; B01L 3/00

(52) U.S. Cl. CPC C12Q 1/6874; B01L 3/502761; B01L 2200/0652; B01L 2300/0654; B01L 2200/16

(57) ABSTRACT

An example of a sequencing nanoparticle includes a core of a negatively chargeable, hydrophobic polymer. Alternating layers of a positively charged acrylamide hydrogel and the negatively charged polymer are positioned on the core, wherein the positively charged acrylamide hydrogel forms an outer layer of the sequencing nanoparticle. A negatively charged primer set is attached to the outer layer.

### CROSS-REFERENCE TO RELATED APPLICATION

This application claims the benefit of U.S. Provisional Application Ser. No. 63/357,479, filed Jun. 30, 2022, the contents of which is incorporated by reference herein in its entirety.

### REFERENCE TO SEQUENCE LISTING

The Sequence Listing submitted herewith is hereby incorporated by reference in its entirety. The name of the file is ILI241B_IP-2337-US_Sequence_Listing.xml, the size of the file is 15,667 bytes, and the date of creation of the file is Jun. 16, 2023.

### BACKGROUND

Various protocols in biological or chemical research involve performing a large number of controlled reactions on local support surfaces or within predefined reaction chambers. The designated reactions may then be observed or detected and subsequent analysis may help identify or reveal properties of chemicals involved in the reaction. In some examples, the controlled reactions generate fluorescence, and thus an optical system may be used for detection.

### SUMMARY

In sequential paired end sequencing, forward strands are generated on a support surface, are sequenced, and are removed, and then reverse strands are generated on the same support surface, are sequenced, and are removed. In the examples disclosed herein, nanoparticles are functionalized with primers that enable sequential paired end sequencing. Methods for making the nanoparticles are disclosed herein, and these methods simplify the fabrication process

### BRIEF DESCRIPTION OF THE FIGURES

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A is a schematic illustration of one example of a sequencing nanoparticle;

FIG. 1B is a schematic illustration of another example of a sequencing nanoparticle;

FIG. 2 is a top view of an example flow cell;

FIG. 3A is an enlarged, cross-sectional view, taken along the 3A-3A line of FIG. 2, depicting one example of the flow cell architecture including the functionalized nanostructures anchored to a lane;

FIG. 3B is an enlarged, cross-sectional view, taken along the 3B-3B line of FIG. 2, depicting another example of the flow cell architecture including the functionalized nanostructures anchored to posts;

FIG. 3C is an enlarged, cross-sectional view, taken along the 3C-3C line of FIG. 2, depicting yet another example of the flow cell architecture including the functionalized nanostructures anchored to depressions;

FIG. 4 is a graph depicting the zeta potential distribution (total counts on the Y axis versus apparent zeta potential, in mV, on the X axis) for poly(lactic acid) nanoparticles and poly(lactic acid) nanoparticles coated with poly(N-(5-azidoacetamidylpentyl)acrylamide-co-acrylamide;

FIG. 5 is a graph depicting the size distribution by intensity (intensity (%) on the Y axis versus size (average diameter, d, in nm) on the X axis) for the poly(lactic acid) nanoparticles and the poly(lactic acid) nanoparticles coated with poly(N-(5-azidoacetamidylpentyl)acrylamide-co-acrylamide; and

FIG. 6 is a graph depicting the zeta potential distribution (total counts on the Y axis versus apparent zeta potential, in mV, on the X axis) for the poly(lactic acid) nanoparticles and poly(lactic acid) nanoparticles prepared with polyethyleneimine.

### DETAILED DESCRIPTION

Each of the sequencing nanoparticles disclosed herein is functionalized with a primer set that enables sequential paired end read sequencing. Because the primer set is attached to the nanoparticles, the sequencing nanoparticles may be used in an off-flow cell library preparation workflow. In these examples, template strand preparation and amplification can take place off of the flow cell, which generates pre-clustered nanoparticles. Then, the pre-clustered nanoparticles may be introduced into, and immobilized onto a surface of, the flow cell for sequencing. Alternatively, the sequencing nanoparticles may be used in an on-flow cell library preparation workflow. In these examples, the (non-clustered) sequencing nanoparticles may be introduced into, and immobilized onto a surface of, the flow cell. In these examples, template strand preparation, amplification, and sequencing all takes place on the flow cell.

The flow cell that is to be used with the sequencing nanoparticles includes capture sites that can anchor the (non-clustered or pre-clustered) sequencing nanoparticles at predetermined locations along the substrate(s) of the flow cell. Because the primer set is part of the sequencing nanoparticles, the flow cell substrate is not exposed to primer grafting processes. As such, the use of the sequencing nanoparticles simplifies the flow cell substrate preparation process.

### Definitions

It is to be understood that terms used herein will take on their ordinary meaning in the relevant art unless specified otherwise. Several terms used herein and their meanings are set forth below.

The singular forms “a,” “an,” and “the” include plural referents unless the context clearly dictates otherwise.

The terms comprising, including, containing and various forms of these terms are synonymous with each other and are meant to be equally broad.

The terms top, bottom, lower, upper, on, etc. are used herein to describe the flow cell and/or the various components of the flow cell. It is to be understood that these directional terms are not meant to imply a specific orientation, but are used to designate relative orientation between components. The use of directional terms should not be interpreted to limit the examples disclosed herein to any specific orientation(s).

The terms first, second, etc. also are not meant to imply a specific orientation or order, but rather are used to distinguish one component from another.

It is to be understood that the ranges provided herein include the stated range and any value or sub-range within the stated range, as if such values or sub-ranges were explicitly recited. For example, a range of about 400 nm to about 1 μm (1000 nm), should be interpreted to include not only the explicitly recited limits of about 400 nm to about 1 μm, but also to include individual values, such as about 708 nm, about 945.5 nm, etc., and sub-ranges, such as from about 425 nm to about 825 nm, from about 550 nm to about 940 nm, etc. Furthermore, when “about” and/or “substantially” are/is utilized to describe a value, they are meant to encompass minor variations (up to +/−10%) from the stated value.

As used herein, the term “attached” refers to the state of two things being joined, fastened, adhered, connected or bound to each other, either directly or indirectly. As examples, bonds that form may be covalent or non-covalent. A covalent bond is characterized by the sharing of pairs of electrons between atoms. A non-covalent bond is a physical bond that does not involve the sharing of pairs of electrons and can include, for example, hydrogen bonds, ionic bonds, van der Waals forces, hydrophilic interactions and hydrophobic interactions.

A “capture site,” as used herein, refers to portion of a flow cell substrate having been modified, chemically, magnetically or electrostatically, that allows for anchoring of a sequencing nanoparticle. In an example, the capture site may include a chemical capture agent, a magnetic capture agent, or an electrostatic capture agent.

A “chemical capture agent” is a material, molecule or moiety that is capable of anchoring to a functional agent of a sequencing nanoparticle via a chemical mechanism. One example chemical capture agent includes a capture nucleic acid (e.g., a capture oligonucleotide) that is complementary to at least a portion of a target nucleic acid attached to a sequencing nanoparticle. Still another example chemical capture agent includes a member of a binding pair that is capable of binding to a second member of a binding pair that is attached to the functionalized nanostructure. Example binding pairs include a NiNTA (nickel-nitrilotriacetic acid) ligand and a histidine tag, or streptavidin or avidin and biotin, etc. Yet another example of the chemical capture agent is a chemical reagent that is capable of forming an electrostatic interaction, a hydrogen bond, or a covalent bond with the sequencing nanoparticle. Covalent bonds may be formed, for example, through thiol-disulfide exchange, click chemistry, Diels-Alder, Michael additions, amine-aldehyde coupling, amine-acid chloride reactions, amine-carboxylic acid reactions, nucleophilic substitution reactions, etc. Some chemical capture agents may be light-triggered, i.e., activated to chemically bind to the chemical capture agent when exposed to light.

The term “depositing,” as used herein, refers to any suitable application technique, which may be manual or automated, and, in some instances, results in modification of the surface properties. Generally, depositing may be performed using vapor deposition techniques, coating techniques, grafting techniques, or the like. Some specific examples include chemical vapor deposition (CVD), spray coating (e.g., ultrasonic spray coating), spin coating, dunk or dip coating, doctor blade coating, puddle dispensing, flow through coating, aerosol printing, screen printing, microcontact printing, inkjet printing, or the like.

As used herein, the term “depression” refers to a discrete concave feature in a substrate having a surface opening that is at least partially surrounded by interstitial region(s) of the substrate. Depressions can have any of a variety of shapes at their opening in a surface including, as examples, round, elliptical, square, polygonal, star shaped (with any number of vertices), etc. The cross-section of a depression taken orthogonally with the surface can be curved, square, polygonal, hyperbolic, conical, angular, etc.

The term “each,” when used in reference to a collection of items, is intended to identify an individual item in the collection, but does not necessarily refer to every item in the collection. Exceptions can occur if explicit disclosure or context clearly dictates otherwise.

As used herein, the term “electrostatic capture agent” refers to a charged material that is capable of electrostatically anchoring a charged sequencing nanoparticle. For the sequencing nanoparticles, the attached primers are negatively charged. As such, positively charged pads, e.g., made of silanes ((3-Aminopropyl)triethoxysilane (APTMS), (3-Aminopropyl)triethoxysilane (APTES), polymers with azide functional groups, polyimines (e.g., polyethyleneimine, polypropylene imine, etc.), and other positively charged materials, may be used as the electrostatic capture agent. Another example of an electrostatic capture agent is an electrode that can attract, when a proper voltage is applied, the charged sequencing nanoparticle.

As used herein, the term “flow cell” is intended to mean a vessel having a flow channel where a reaction can be carried out, an inlet for delivering reagent(s) to the flow channel, and an outlet for removing reagent(s) from the flow channel. In some examples, the flow cell enables the detection of the reaction that occurs in the chamber. For example, the flow cell may include one or more transparent surfaces allowing for the optical detection of arrays, optically labeled molecules, or the like within the flow channel.

As used herein, a “flow channel” or “channel” may be an area defined between two bonded components, which can selectively receive a liquid sample. In some examples, the flow channel may be defined between a substrate and a lid, and thus may be in fluid communication with one or more depressions defined in the substrate or capture sites positioned on the substrate. The flow channel may also be defined between two substrate surfaces that are bonded together.

A “functional agent” is a material, molecule or moiety that is capable of anchoring to a chemical capture site of a flow cell via a chemical mechanism. One example functional agent includes a target nucleic acid that is complementary to a capture nucleic acid (e.g., a capture oligonucleotide) on the flow cell. Still another example functional agent includes a member of a binding pair that is capable of binding to a second member of a binding pair that is attached to the flow cell.

As used herein, the term “interstitial region” refers to an area, e.g., of a substrate that separates depressions or capture sites. For example, an interstitial region can separate one depression of an array from another depression of the array. The two depressions that are separated from each other can be discrete, i.e., lacking physical contact with each other. In many examples, the interstitial region is continuous whereas the depressions are discrete, for example, as is the case for a plurality of depressions defined in an otherwise continuous surface. The separation provided by an interstitial region can be partial or full separation. Interstitial regions may have a surface material that differs from the surface material of the depressions or of the capture site material.

As used herein, the term “magnetic capture agent” refers to a magnetic material that is capable of magnetically anchoring a sequencing nanoparticle. Example magnetic capture agents include ferromagnetic materials and ferrimagnetic materials.

As used herein, the term “mechanism” refers to a functional agent, a magnetic material, or a charged species (e.g., primers of the primer set) that is incorporated into or attached to the sequencing nanoparticle in order to render the sequencing nanoparticle capable of anchoring to a capture site in a flow cell.

As used herein, a “nucleotide” includes a nitrogen containing heterocyclic base, a sugar, and one or more phosphate groups. Nucleotides are monomeric units of a nucleic acid sequence. In ribonucleic acids (RNA), the sugar is a ribose, and in deoxyribonucleic acids (DNA), the sugar is a deoxyribose, i.e., a sugar lacking a hydroxyl group that is present at the 2′ position in ribose. The nitrogen containing heterocyclic base (i.e., nucleobase) can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. A nucleic acid analog may have any of the phosphate backbone, the sugar, or the nucleobase altered. Examples of nucleic acid analogs include, for example, universal bases or phosphate-sugar backbone analogs, such as peptide nucleic acid (PNA).

The term “orthogonal,” when used to describe two functional groups or two cleaving chemistries means that the groups or chemistries are different from each other. Orthogonal functional groups are capable of reacting with different functional groups, e.g., an azide may be reacted with an alkyne or DBCO (dibenzocyclooctyne) while an amino may be reacted with an activated carboxylate group or an N-hydroxysuccinimide (NHS) ester. Orthogonal cleaving chemistries are susceptible to different cleaving agents so that the first cleaving chemistry is unaffected when exposed to the cleaving agent for the second cleaving chemistry, and the second cleaving chemistry is unaffected when exposed to the cleaving agent for the first cleaving chemistry.

As used herein, the term “primer” is defined as a single stranded nucleic acid sequence (e.g., single strand DNA). Some primers are part of a primer set, which serve as a starting point for template amplification and cluster generation. The 5′ terminus of each primer in a primer set may be modified to allow a coupling reaction with a functional group of a polymer chain. Other primers, referred to herein as sequencing primers, serve as a starting point for DNA synthesis. In an example, the primer length can be any number of bases long and can include a variety of non-natural nucleotides. In an example, the sequencing primer is a short strand, ranging from 10 to 60 bases, or from 20 to 40 bases.

The term “primer set” refers to a pair of primers that together enable the amplification of a template nucleic acid strand (also referred to herein as a library template). Opposed ends of the template strand include adapters to hybridize to the respective primers in a set.

The term “substrate” refers to a structure upon which various components of the flow cell (e.g., capture sites, etc.) may be added. The substrate may be a wafer, a panel, a rectangular sheet, a die, or any other suitable configuration. The substrate is generally rigid and is insoluble in an aqueous liquid. The substrate may be inert to the chemistry of the capture sites and the sequencing nanoparticles. For example, a substrate can be inert to chemistry used to attach the sequencing nanoparticle(s), used in sequencing reactions, etc. The substrate may be a single layer structure, or a multi-layered structure (e.g., including a support and a patterned resin on the support). Examples of suitable substrates will be described further herein.

Sequencing Nanoparticles and Methods of Making

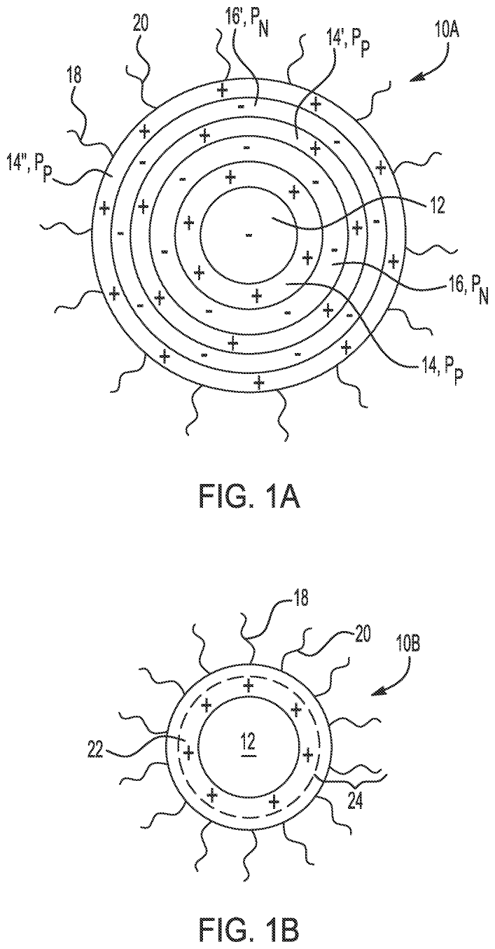

Examples of the sequencing nanoparticles 10A, 10B are shown in FIG. 1A and FIG. 1B. Each of the sequencing nanoparticles 10A, 10B is formed using layer-by-layer processing.

The sequencing nanoparticle 10A shown in FIG. 1A includes a core 12 of a negatively chargeable, hydrophobic polymer P_{N}; alternating layers 14, 16 of a positively charged acrylamide hydrogel P_{P} and the negatively chargeable, hydrophobic polymer P_{N} positioned on the core 12, wherein the positively charged acrylamide hydrogel P_{P} forms an outer layer (e.g., 14″, P_{P} in FIG. 1A); and a negatively charged primer set, including primers 18, 20, attached to the outer layer 14″, P_{P}.

The negatively chargeable, hydrophobic polymer P_{N} used to form the core 12 and the layers 16, 16′ is a synthetic polyester polymer having negatively chargeable atoms or functional groups at the chain ends. In an example, the negatively chargeable, hydrophobic polymer P_{N} is selected from the group consisting of poly(lactic acid) (PLA), poly(lactic-co-glycolic acid) (PLGA), poly(ε-caprolactone) (PCL), and poly(glycolic acid) (PGA). The negatively chargeable atoms or functional groups (e.g., carboxylic acid groups) become charged when exposed to aqueous media, e.g., during the flash nanoprecipitation process disclosed herein.

The positively charged acrylamide hydrogel P_{P} is a gel material that can swell when liquid is taken up and can contract when liquid is removed, e.g., by drying. In each of the examples disclosed herein, the positively charged acrylamide hydrogel P_{P} is an acrylamide co-polymer having positively charged atoms or functional groups in the side chains.

Each example of the acrylamide co-polymer includes three different monomers, one of which includes a terminal functional group that is capable of attaching the primers 18, 20 to the hydrogel P_{P} and another of which imparts the positive charge to the hydrogel P_{P}. The positive charge may be introduced to the monomer represented at “o” in structure (I) below during polymerization by using a co-initiator, such as N,N,N′,N′-Tetramethyl ethylenediamine (TEMED), or another non-cross-linking alkylamine).

In one example, the acrylamide co-polymer is represented by the following structure (I):

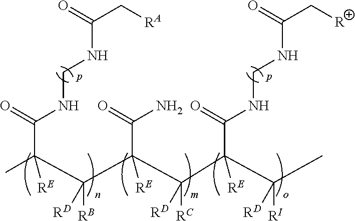

wherein: R^{A} is the terminal functional group that is capable of attaching the primers to the positively charged acrylamide hydrogel P_{P}, and is selected from the group consisting of an azide group, an amino group, an alkyne group, an aldehyde group, a hydrazine group, a carboxyl group, a hydroxyl group, a tetrazole group, a tetrazine group, a nitrile oxide group, a nitrone group, a thiol group, and combinations thereof; R^{B}, R^{C}, R^{D}, R^{E} and R^{I} are each independently selected from the group consisting of H and an alkyl; R⁺ is the terminal functional group that imparts the positive charge to the positively charged acrylamide hydrogel P_{P}, and is a quaternary ammonium cation, such as N(CH₃)₃; —(CH₂)ₚ— and —(CH₂)_{p′}— can be optionally substituted; p and p′ are each an integer in the range of 1 to 50; n is an integer in the range of 1 to 50,000; m is an integer in the range of 1 to 100,000; and o is an integer in the range of 1 to 50,000. As such, some of the terminal functional group R^{A} may attach the primers and others of the terminal functional group R^{A} may attach to an underlying substrate.

One specific example of the acrylamide co-polymer represented by structure (II):

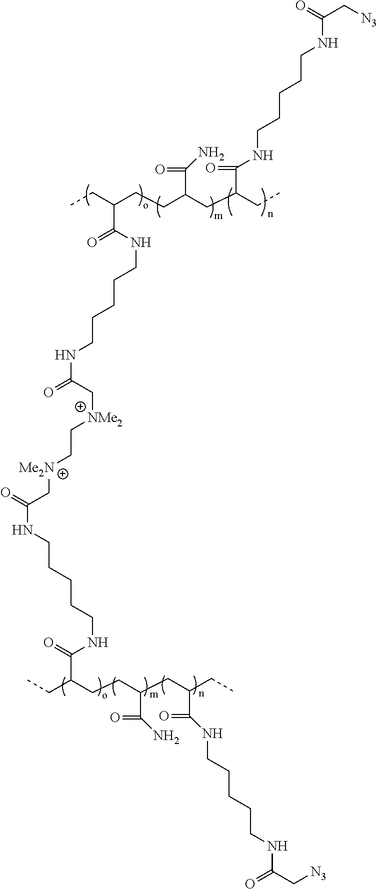

In structure (II): R^{A} is an azide group; R^{B}, R^{D}, R^{D}, R^{E} and R^{I} are each H; R⁺ is N(CH₃)₃; p and p′ are each 5; and n, m, and o are as defined for structure (I). As depicted in structure (II), the R⁺ groups may cross-link with one another. In one example, the extent of cross-linking is less than 1%.

One of ordinary skill in the art will recognize that the arrangement of the recurring “n” and “m” and “o” features in structures (I) and (II) are representative, and the monomeric subunits may be present in any order in the polymer structure (e.g., random, block, patterned, or a combination thereof). In some examples, the acrylamide co-polymer of structure (I) or (II) is a linear polymer. In some other examples, the acrylamide co-polymer of structure (I) or (II) is a lightly cross-linked polymer.

The molecular weight of the acrylamide co-polymer represented by structure (I) or (II) may range from about 5 kDa to about 1500 kDa or from about 10 kDa to about 1000 kDa, or may be, in a specific example, about 312 kDa.

In other examples, the positively charged acrylamide hydrogel P_{P} may be a variation of structure (I). In one example, the acrylamide unit may be replaced with N,N-dimethylacrylamide

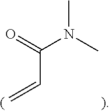

In this example, the acrylamide unit in structure (I) may be replaced with,

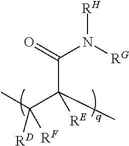

where R^{D}, R^{E}, R^{F} and R^{I} are each H or a C1-C6 alkyl, and R^{G} and R^{H} are each a C1-C6 alkyl (instead of H as is the case with the acrylamide). In this example, q may be an integer in the range of 1 to 100,000. In another example, the N,N-dimethylacrylamide may be used in addition to the acrylamide unit. In this example, structure (I) may include

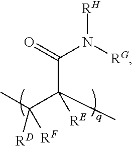

in addition to the recurring “n” and “m” and “o” features, where R^{D}, R^{E}, R^{F} and R^{I} are each H or a C1-C6 alkyl, and R^{G} and R^{H} are each a C1-C6 alkyl. In this example, q may be an integer in the range of 1 to 100,000.

As still another example of the polymeric hydrogel, the recurring “n” feature in structure (I) may be replaced with a monomer including a heterocyclic azido group having structure (III):

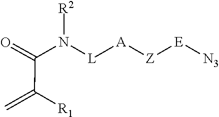

wherein: R₁ is H or a C1-C6 alkyl; R₂ is H or a C1-C6 alkyl; L is a linker including a linear chain with 2 to 20 atoms selected from the group consisting of carbon, oxygen, and nitrogen and 10 optional substituents on the carbon and any nitrogen atoms in the chain; E is a linear chain including 1 to 4 atoms selected from the group consisting of carbon, oxygen and nitrogen, and optional substituents on the carbon and any nitrogen atoms in the chain; A is an N substituted amide with an H or a C1-C4 alkyl attached to the N; and Z is a nitrogen containing heterocycle. Examples of Z include 5 to 10 carbon-containing ring members present as a single cyclic structure or a fused structure. Some specific examples of Z include pyrrolidinyl, pyridinyl, or pyrimidinyl.

As still another example, the positively charged acrylamide hydrogel P_{P} may include a recurring unit of each of structure (IV) and (V):

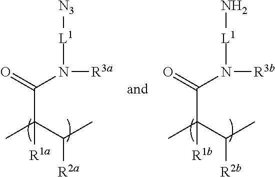

wherein: each of R^{1a}, R^{2a}, R^{1b} and R^{2b} is independently selected from H, an alkyl or a phenyl; each of R^{3a} and R^{3b} is independently selected from hydrogen, an alkyl, a phenyl, or a C7-C14 aralkyl; and each L1 and L2 is independently selected from an alkylene linker or a heteroalkylene linker.

In any example of the positively charged acrylamide hydrogel P_{P}, the first monomer (recurring feature “n”) makes up from about 0.1% to about 20% of the co-polymer; the second monomer (recurring feature “m” or “q”) makes up from about 60% to less than 100% of the co-polymer; and the third monomer (recurring feature “o”) makes up from about 0.1% to about 20% of the co-polymer. In another specific example, the first monomer makes up from about 5% to about 15% of the co-polymer; the second monomer makes up from makes up from about 70% to about 90% of the co-polymer; and the third monomer makes up from makes up from about 5% to about 15% of the co-polymer.

As mentioned, the sequencing nanoparticle 10A includes a sequential paired end sequencing primer set that includes two different primers 18, 20. In one example of sequential paired end sequencing, the respective forward strands that are generated (e.g., via amplification) are sequenced and removed, and then the respective reverse strands are generated, sequenced, and removed.

The primers 18, 20 include a single strand of DNA. In the DNA structure, each phosphate group includes one negatively charged oxygen atom, which renders the entire strand negatively charged.

As examples, the primers 18, 20 in the primer set may include P5 and P7 primers, P15 and P7 primers, or any combination of the PA primers, the PB primers, the PC primers, and the PD primers set forth herein. As example combinations, the primer set may include any two PA, PB, PC, and PD primers, or any combination of one PA primer and one PB, PC, or PD primer, or any combination of one PB primer and one PC or PD primer, or any combination of one PC primer and one PD primer.

Examples of P5 and P7 primers are used on the surface of commercial flow cells sold by Illumina Inc. for sequencing, for example, on HISEQ™, HISEQX™, MISEQ™, MISEQDX™, MINISEQ™, NEXTSEQ™, NEXTSEQDX™, NOVASEQ™, ISEQ™, GENOME ANALYZER™, and other instrument platforms.

The P5 primer is:

P5: 5′ → 3′
(SEQ. ID. NO. 1)
AATGATACGGCGACCACCGAGAUCTACAC

The P7 primer may be any of the following:

P7 #1: 5′ → 3′
(SEQ. ID. NO. 2)
CAAGCAGAAGACGGCATACGAnAT
P7 #2: 5′ → 3′
(SEQ. ID. NO. 3)
CAAGCAGAAGACGGCATACnAGAT
P7 #3: 5′ → 3′
(SEQ. ID. NO. 4)
CAAGCAGAAGACGGCATACnAnAT

where “n” is 8-oxoguanine in each of these sequences.
The P15 primer is:

P15: 5′ → 3′
(SEQ. ID. NO. 5)
AATGATACGGCGACCACCGAGAnCTACAC

where “n” is allyl-T (a thymine nucleotide analog having an allyl functionality).
The other primers (PA-PD) mentioned above include:

PA 5′ → 3′
(SEQ. ID. NO. 6)
GCTGGCACGTCCGAACGCTTCGTTAATCCGTTGAG
cPA (PA′) 5′ → 3′
(SEQ. ID. NO. 7)
CTCAACGGATTAACGAAGCGTTCGGACGTGCCAGC
PB 5′ → 3′
(SEQ. ID. NO. 8)
CGTCGTCTGCCATGGCGCTTCGGTGGATATGAACT
cPB (PB′) 5′ → 3′
(SEQ. ID. NO. 9)
AGTTCATATCCACCGAAGCGCCATGGCAGACGACG
PC 5′ → 3′
(SEQ. ID. NO. 10)
ACGGCCGCTAATATCAACGCGTCGAATCCGCAACT
cPC (PC′) 5′ → 3′
(SEQ. ID. NO. 11)
AGTTGCGGATTCGACGCGTTGATATTAGCGGCCGT
PD 5′ → 3′
(SEQ. ID. NO. 12)
GCCGCGTTACGTTAGCCGGACTATTCGATGCAGC
cPD (PD′) 5′ → 3′
(SEQ. ID. NO. 13)
GCTGCATCGAATAGTCCGGCTAACGTAACGCGGC.

While not shown in the example sequences for PA-PD, it is to be understood that any of these primers may include a cleavage site, such as uracil, 8-oxoguanine, allyl-T, etc. at any point in the strand, as long as the cleavage sites of the primers 18 and 20 are orthogonal (i.e., the cleaving chemistry of the primer 18 is different than the cleaving chemistry for the primer 20, and thus the two primers 18, 20 are susceptible to different cleaving agents).

Each of the primers 18, 20 disclosed herein may also include a polyT sequence at the 5′ end of the primer sequence. In some examples, the polyT region includes from 2 T bases to 20 T bases. As specific examples, the polyT region may include 3, 4, 5, 6, 7, or 10 T bases.

The 5′ terminal end of the primers 18, 20 will vary depending upon the chemistry of the positively charged acrylamide hydrogel P_{P}. As two examples, the 5′ end functional groups may be a terminal alkyne (e.g., hexynyl) or an internal alkyne, where the alkyne is part of a cyclic compound (e.g., bicyclo[6.1.0]nonyne (BCN)). The terminal alkynes can attach to azide groups on the positively charged acrylamide hydrogel P_{P}. In another example, the primers 18, 20 may include an alkene at the 5′ terminus, which can react with reactive thiol groups on the positively charged acrylamide hydrogel P_{P}. In still other specific examples, succinimidyl (NHS) ester terminated primers may be reacted with amine groups on the positively charged acrylamide hydrogel P_{P}; aldehyde terminated primers may be reacted with hydrazine groups on the positively charged acrylamide hydrogel P_{P}; or azide terminated primers may be reacted with an alkyne or DBCO (dibenzocyclooctyne) on the positively charged acrylamide hydrogel P_{P}.

A method for making the sequencing nanoparticle 10A includes generating a nanoparticle (i.e., the core 12) of the negatively chargeable, hydrophobic polymer P_{N}; in a layer-by-layer fashion, sequentially forming layers 14, 16, 14′, 16′, 14″ of the positively charged acrylamide hydrogel P_{P} and of the negatively chargeable, hydrophobic polymer P_{N} on the nanoparticle 12 to form a coated nanoparticle until i) a particle size of a dry form of the coated nanoparticle ranges from about 200 nm to about 1 μm, and ii) the positively charged acrylamide hydrogel P_{P} forms an outer layer e.g., layer 14″) of the coated nanoparticle; and grafting a negatively charged primer set (e.g., primers 18, 20) to the outer layer 14″. In another example of the method, the nanoparticle (i.e., the core 12) of the negatively chargeable, hydrophobic polymer P_{N} and then a single layer of the positively charged acrylamide hydrogel P_{P} is coated on the core 12.

Generating the nanoparticle core 12 of the negatively chargeable, hydrophobic polymer P_{N} involves flash nanoprecipitation. With flash nanoprecipitation, the negatively chargeable, hydrophobic polymer P_{N} is dissolved in an organic solvent. The selection of the organic solvent will depend upon the negatively chargeable, hydrophobic polymer P_{N} that is used. Any water-miscible organic solvent may be used that is capable of dissolving the negatively chargeable, hydrophobic polymer P_{N}. In an example, the organic solvent is acetone, ethanol, tetrahydrofuran (THF), or isopropyl alcohol (IPA). In addition to being able to dissolve the negatively chargeable, hydrophobic polymer P_{N}, the selected organic solvent may also affect the surface properties of the nanoparticle core 12. In particular, the physical properties of the organic solvents (e.g., dielectric constant and solubility) may affect the following properties of the nanoparticle core 12: size, surface charge, and porosity. The organic solvent can also affect the particle formation, solvent evaporation (in subsequent processing step(s)), core size, colloidal stability, and freeze-drying ability. In an example, nanoparticle cores 12 with desirable attributes are obtained when the negatively chargeable, hydrophobic polymer P_{N} concentration in the solution ranges from about 2 mg/mL to about 30 mg/mL. This concentration range may be particularly suitable for PLA in acetone.

The solution of the negatively chargeable, hydrophobic polymer P_{N} dissolved in the organic solvent is introduced into a non-solvent of the negatively chargeable, hydrophobic polymer P_{N}. Alternatively, the non-solvent may be added to the solution of the negatively chargeable, hydrophobic polymer P_{N}. The non-solvent is not capable of dissolving the negatively chargeable, hydrophobic polymer P_{N}, and thus the selection of the non-solvent will depend upon the negatively chargeable, hydrophobic polymer P_{N} that is used. In an example, the non-solvent is water (e.g., deionized water). The non-solvent may also include a stabilizing agent. Suitable stabilizing agents are surfactants (amphiphilic molecules characterized by a hydrophilic head group (ionic or non-ionic) and a hydrophobic tail). The amphiphilic nature of the surfactant can stabilize the hydrophobic nanoparticle core 12 in the aqueous media. In particular, hydrophobic regions of the surfactant interact with the nanoparticle core 12 surface and hydrophilic regions of the surfactant interact with water. One suitable class of stabilizing agents is poloxamers, which are block copolymers consisting of hydrophilic poly(ethylene oxide) (PEO) and hydrophobic poly(propylene oxide) (PPO)), and which are commercially available under the tradename PLURONICS® from BASF Corp.

The solution of the negatively chargeable, hydrophobic polymer P_{N} dissolved in the organic solvent is introduced into the non-solvent or the non-solvent is introduced into the solution of the negatively chargeable, hydrophobic polymer P_{N} dissolved in the organic solvent so that the volume ratio of the organic phase to the aqueous phase ranges from 1:2 to 1.4. The mixture is stirred rapidly for up to 10 minutes, which stabilizes the negatively chargeable, hydrophobic polymer P_{N} in the form of nanosized particles (the cores 12).

The negatively chargeable, hydrophobic polymer P_{N} dissolved in the solvent forms the diffusing phase. In one example, the organic diffusing phase is added dropwise (total volume added ranging from about 5 mL to about 10 mL) to the aqueous dispersing phase (total volume ranging from about 10 mL to about 20 mL) using a syringe, dropper, or other like dispensing apparatus under moderate magnetic stirring or other moderate agitation. The nanoparticle cores 12 form within minutes of the diffusing phase being added to the dispersing phase. The agitation helps to ensure that macroscopic aggregates do not form. This process is performed at room temperature (e.g., from about 19° C. to about 23° C.).

The charge at the surface of the core 12 depends upon environment (e.g., pH, presence of salt, etc.) in which the cores 12 are formed. For example, PLA carboxylates are negatively charged at a working pH of about 7.

The solvent and non-solvent of the mixture are evaporated, leaving a plurality of the nanoparticle cores 12. Evaporation may be performed under vacuum using a rotary evaporator. The water bath of the rotary evaporator may be heated to about 40° C. to accelerate the solvent evaporation. The nanoparticle cores 12 have an intensity particle size distribution (determined using Dynamic Light Scattering) ranging from about 95 nm to about 190 nm.

The nanoparticle cores 12 are then exposed to layer-by-layer assembly to generate the desired number of oppositely charged layers 14, 16, 14′, 16′, 14″. To generate the first layer 14, the negatively charged nanoparticle cores 12 are exposed to the positively charged acrylamide hydrogel P_{P}, e.g., using immersion, spin coating, or spray coating. The negatively charged nanoparticle cores 12 absorb the positively charged acrylamide hydrogel P_{P}. The layer 14 of the positively charged acrylamide hydrogel P_{P} is formed on the surface of the negatively charged nanoparticle core 12, rendering the coated particle positively charged at its surface. The coated particle may be washed or exposed to a purification process (e.g., centrifugation) to remove excess positively charged acrylamide hydrogel P_{P}.

To generate the second layer 16, the coated particles (i.e., the negatively charged nanoparticle cores 12 with the layer 14 of the positively charged acrylamide hydrogel P_{P} at their surfaces) are exposed to the negatively chargeable, hydrophobic polymer P_{N}, e.g., using immersion or dip coating, spin coating, spray coating, or flow through coating using a flow through reactor. These processes may be performed in the presence of water to generate negative charges on the polymer P_{N}. The positively charged acrylamide hydrogel P_{P} absorbs the (now) negatively charged polymer P_{N}. The layer 16 of the negatively charged polymer P_{N} is formed on the surface of the positively charged layer 14, rendering the coated particle negatively charged at its surface. The coated particle may be washed or exposed to a purification process (e.g., centrifugation) to remove excess negatively chargeable, hydrophobic polymer P_{N}.

The layer-by-layer coating process may be repeated as many times as desired to generate alternating layers 14, 16, 14′, 16′, 14″. The layer-by-layer coating process is controlled so that i) the resulting coated particle (in its dry form) has a particle size ranging from about 200 nm to about 1 μm, and ii) the positively charged acrylamide hydrogel P_{P} forms the outermost layer (e.g., layer 14″). The positively charged acrylamide hydrogel P_{P} at the surface of the coated particle can be in a dry state or can be in a swollen state, where it uptakes liquid. When the layer 14″ is in the swollen state, the particle size may be greater than the range provided herein.

The primers 18, 20 may then be grafted to the outer layer 14″ of the positively charged acrylamide hydrogel P_{P}. Grafting may be accomplished by flow through dunk coating, spray coating, puddle dispensing, or by another suitable method. Each of these example techniques may utilize a primer solution or mixture, which may include the primer(s) 18, 20, water, a buffer, and a catalyst. In one example, copper-catalyzed azide-alkyne cycloaddition is used. With any of the grafting methods, the primers 18, 20 attach to the reactive groups of the positively charged acrylamide hydrogel P_{P}.

The sequencing nanoparticle 10B shown in FIG. 1B includes a core 12 of a negatively chargeable, hydrophobic polymer P_{N}; a positively charged polymer coating 22 attached to the core 12; and a negatively charged pre-grafted acrylamide hydrogel 24 attached to the positively charged polymer coating 22.

The core 12 of the sequencing nanoparticle 10B may be any of the examples described in reference to FIG. 1A. In one example, the negatively chargeable, hydrophobic polymer P_{N} used to form the core 12 is selected from the group consisting of poly(lactic acid) (PLA), poly(lactic-co-glycolic acid) (PLGA), poly(ε-caprolactone) (PCL), and poly(glycolic acid) (PGA). In these examples, the core 12 does not exhibit the negative charges, as the particles are not formed in the aqueous medium (the method of which is described below).

In this example, the positively charged polymer coating 22 at the surface of the core 12 is polyethyleneimine (PEI), chitosan, poly(L-lysine), or poly(diallyldimethylammonium chloride) poly(allylamine hydrochloride) (PAH).

The negatively charged pre-grafted acrylamide hydrogel 24 may be any example of the positively charged acrylamide hydrogel P_{P} with the primers 18, 20 grafted thereto. The overall charge of the pre-grafted acrylamide hydrogel 24 is negative due to the primers 18, 20. Any example of the positively charged acrylamide hydrogel P_{P} and the primers 18, 20 may be used in this example. As one example, the pre-grafted acrylamide hydrogel is poly(N-(5-azidoacetamidylpentyl)acrylamide-co-acrylamide having forward and reverse primers (e.g., P5 and P7) grafted thereto.

A method for making the sequencing nanoparticle 10B includes grafting a negatively charged primer set 18, 20 to an acrylamide hydrogel (e.g., positively charged acrylamide hydrogel P_{P}), thereby generating a negatively charged pre-grafted acrylamide hydrogel 24; generating a nanoparticle having a core 12 and a positively charged polymer coating 22 at a surface of the core 12; and attaching the negatively charged pre-grafted acrylamide hydrogel 24 to the positively charged polymer coating 22.

The positively charged acrylamide hydrogel P_{P} may be polymerized using monomers that will generate the desired repeat units and any suitable polymerization technique. The primers 18, 20 may be grafted to the acrylamide hydrogel as described herein to form the negatively charged pre-grafted acrylamide hydrogel 24.

The nanoparticle having the core 12 and the positively charged polymer coating 22 at the surface involves a modified flash nanoprecipitation process. In this modified process, polyethyleneimine or another positively charged polymer (e.g., chitosan, poly(L-lysine), or poly(diallyldimethylammonium chloride) poly(allylamine hydrochloride) (PAH)) is used as the non-solvent phase during precipitation of the core 12.

At the outset of the modified flash nanoprecipitation process, the negatively chargeable, hydrophobic polymer P_{N} is dissolved in any example of the organic solvent disclosed herein. In this example, the solution of the negatively chargeable, hydrophobic polymer P_{N} dissolved in the organic solvent is introduced into the polyethyleneimine or other positively charged polymer or the polyethyleneimine or other positively charged polymer is introduced into the solution of the negatively chargeable, hydrophobic polymer P_{N} dissolved in the organic solvent. The solution of the negatively chargeable, hydrophobic polymer P_{N} dissolved in the organic solvent is introduced into the polyethyleneimine or other positively charged polymer, or the polyethyleneimine or other positively charged polymer is introduced into the solution of the negatively chargeable, hydrophobic polymer P_{N} dissolved in the organic solvent so that the volume ratio of the negatively chargeable, hydrophobic organic phase to the positively charged organic phase ranges from 1:2 to 1:4. The mixture is stirred rapidly for up to 10 minutes, which stabilizes the negatively chargeable, hydrophobic polymer P_{N} in nanosized particles (the cores 12) and allows the polyethyleneimine or other positively charged polymer to coat the surface of the cores 12.

In this example method, the negatively chargeable, hydrophobic polymer P_{N} dissolved in the solvent forms the diffusing phase, and the cationic polymer formed the dispersing phase. In one example, the organic diffusing phase is added dropwise (total volume added ranging from about 5 mL to about 10 mL) to the cationic dispersing phase (total volume ranging from about 10 mL to about 20 mL) using a syringe, dropper, or other like dispensing apparatus under moderate magnetic stirring or other moderate agitation. The nanoparticle cores 12 form within minutes of the diffusing phase being added to the dispersing phase, and the cationic polymer strands coat the surface of the cores 12. The agitation helps to ensure that macroscopic aggregates do not form. This process is performed at room temperature.

The solvent of the mixture is evaporated as described herein, leaving a plurality of positively charged and coated nanoparticles.

The positively charged and coated nanoparticles are then exposed to the negatively charged pre-grafted acrylamide hydrogel 24, e.g., using immersion, spin coating, or spray coating. The positively charged and coated nanoparticles absorb the negatively charged pre-grafted acrylamide hydrogel 24. The layer 24 of the negatively charged pre-grafted acrylamide hydrogel 24 is formed on the surface of the positively charged polymer coating 22, rendering the coated particle negatively charged at its surface. The coated particle 10B may be washed or exposed to a purification process (e.g., centrifugation) to remove excess negatively charged pre-grafted acrylamide hydrogel 24.

Each of the sequencing nanoparticles 10A, 10B is also capable of anchoring to a capture site on a flow cell substrate. As such, the sequencing nanoparticles 10A, 10B include some mechanism that is capable of attaching to the capture site. The mechanism may be chemical (e.g., a functional agent), electrostatic, or magnetic.

In some examples, the mechanism is a component of the sequencing nanoparticles 10A, 10B that enables it to be anchored without further functionalization. For example, when the sequencing nanoparticles 10A, 10B include a magnetic material as part of the core 12, the sequencing nanoparticles 10B may be anchored to a magnetic capture agent on the flow cell substrate without further functionalization. In this particular example, a mini-emulsion polymerization process may be used to form magnetic (Fe₃O₄) polystyrene particles. Oppositely charged polyelectrolytes can then be alternatingly absorbed at the surface of the particles using the layer-by-layer process disclosed herein. For another example, when the positive charges at the surface of the sequencing nanoparticles 10A, 10B are used as the mechanism, the sequencing nanoparticles 10B may be anchored to an electrostatic capture agent on the flow cell substrate.

In other examples, the mechanism is a functional agent that is added to the sequencing nanoparticle 10A, 10B that enables it to be anchored on the flow cell substrate. As one example, a target nucleic acid, that is complementary to a capture oligonucleotide on the flow cell substrate, may be grafted to the outer layer 14″, P_{P} or to the negatively charged pre-grafted acrylamide hydrogel 24. As other examples, a functional group for covalent attachment or a member of a binding pair may be grafted to or chemically introduced to the outer layer 14″, P_{P} or to the negatively charged pre-grafted acrylamide hydrogel 24.

Flow Cells for Use with the Sequencing Nanoparticles

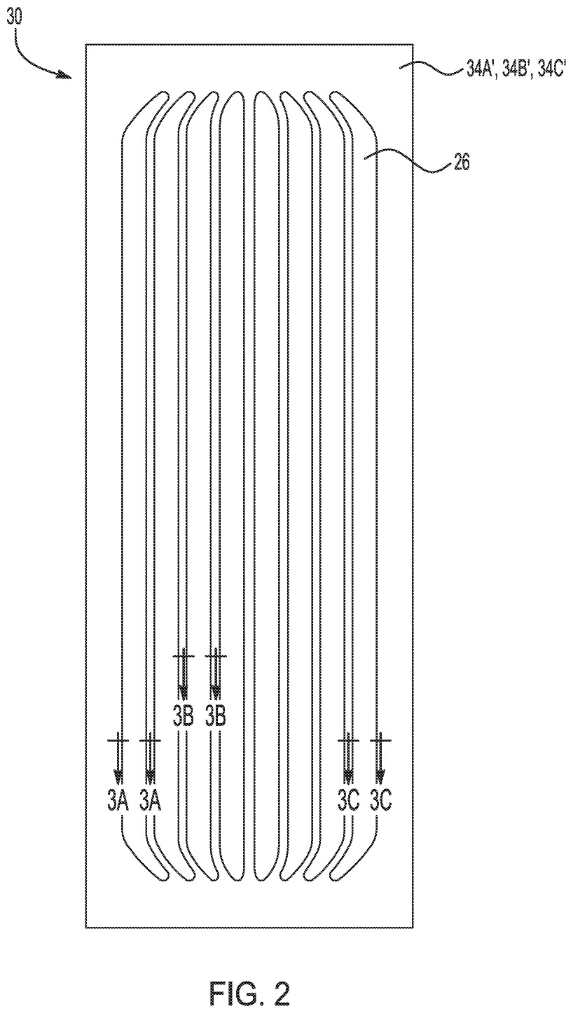

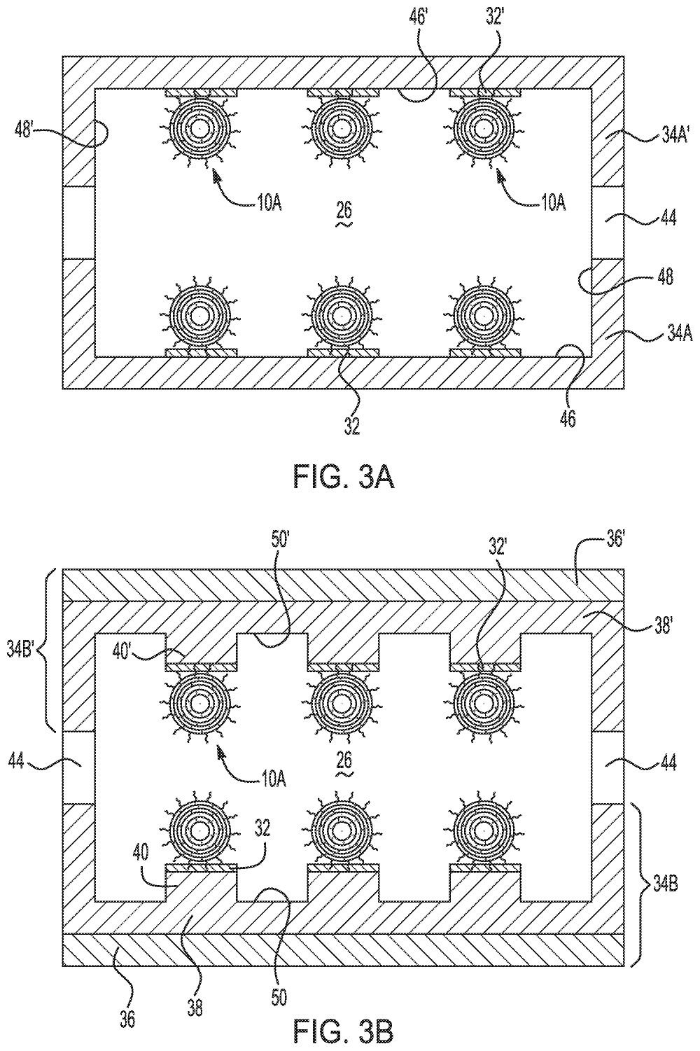

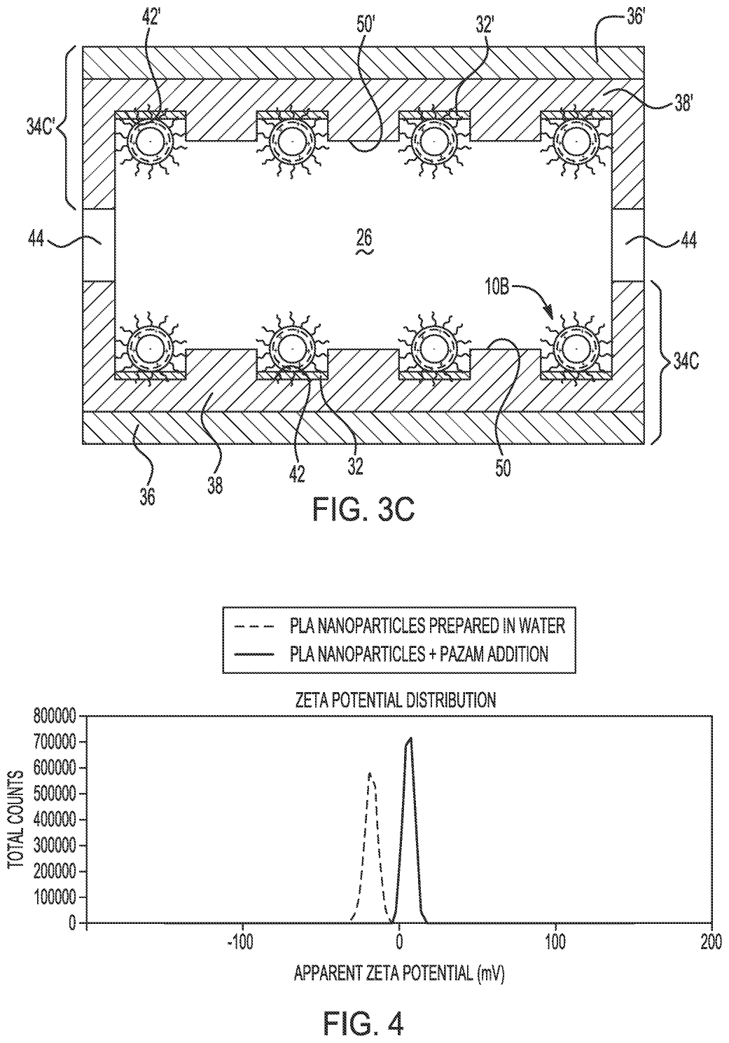

The sequencing nanoparticles 10A, 10B may be used with any flow cell 30 (FIG. 2) that includes capture sites 32, 32′ (FIG. 3A, FIG. 3B, FIG. 3C). An example of the flow cell 30 is depicted from the top view in FIG. 2, and different examples of the flow cell architecture, including different configurations of the capture sites 32, 32′, are shown in FIG. 3A, FIG. 3B, and FIG. 3C.

A top view of an example of the flow cell 30 is shown in FIG. 2. As will be discussed in reference to FIG. 3A, FIG. 3B, and FIG. 3C, some examples of the flow cell 30 include two opposed substrates 34A, 34A′ or 34B, 34B′ or 34C, 34C′, each of which is configured with capture sites 32, 32′. In these examples, a flow channel 26 is defined between the two opposed substrates 34A, 34A′ or 34B, 34B′ or 34C, 34C′. In other examples, the flow cell 30 includes one substrate 34A or 34B or 34C configured with capture sites 32 and a lid attached to the substrate 34A or 34B or 34C. In these examples, the flow channel 26 is defined between the substrate 34A or 34B or 34C and the lid. In still other examples, the flow cell 30 includes one substrate 34A or 34B or 34C that is used in an open configuration.

Different substrates 34A, 34A′ or 34B, 34B′ or 34C, 34C′ are shown in FIG. 3A and FIGS. 3B and 3C.

In the example shown in FIG. 3A, the substrates 34A, 34A′ are single layered structures. Examples of suitable single layered structures for the substrate 34A, 34A′ include epoxy siloxane, glass, modified or functionalized glass, plastics (including acrylics, polystyrene and copolymers of styrene and other materials, polypropylene, polyethylene, polybutylene, polyurethanes, polytetrafluoroethylene (such as TEFLON® from Chemours), cyclic olefins/cyclo-olefin polymers (COP) (such as ZEONOR® from Zeon), polyimides, etc.), nylon (polyamides), ceramics/ceramic oxides, silica, fused silica, or silica-based materials, aluminum silicate, silicon and modified silicon (e.g., boron doped p+ silicon), silicon nitride (Si₃N₄), silicon oxide (SiO₂), tantalum pentoxide (Ta₂O₅) or other tantalum oxide(s) (TaOₓ), hafnium oxide (HfO₂), carbon, metals, inorganic glasses, or the like.

In the examples shown in FIG. 3B and FIG. 3C, the substrates 34B, 34B′ and 34C, 34C′ are multi-layered structures. The multi-layered structures of the substrates 34B, 34B′ and 34C, 34C′ include a base support 36 or 36′ and a patterned material 38 or 38′ on the base support 36, 36′.

The base support 36, 36′ may be any of the examples set forth herein for the single layered structure of the substrate 34A, 34A′.

The patterned material 38, 38′ may be any material that is capable of being patterned with posts 40, 40′ (FIG. 3B) or depressions 42, 42′ (FIG. 3C).

In an example, the patterned material 38, 38′ may be an inorganic oxide that is selectively applied to the base support 36, 36′, e.g., via vapor deposition, aerosol printing, or inkjet printing, in the desired pattern. Examples of suitable inorganic oxides include tantalum oxide (e.g., Ta₂O₅), aluminum oxide (e.g., Al₂O₃), silicon oxide (e.g., SiO₂), hafnium oxide (e.g., HfO₂), etc.

In another example, the patterned material 38, 38′ may be a resin matrix material that is applied to the base support 36, 36′ and then patterned. Suitable deposition techniques include chemical vapor deposition, dip coating, dunk coating, spin coating, spray coating, puddle dispensing, ultrasonic spray coating, doctor blade coating, aerosol printing, screen printing, microcontact printing, etc. Suitable patterning techniques include photolithography, nanoimprint lithography (NIL), stamping techniques, embossing techniques, molding techniques, microetching techniques, printing techniques, etc. Some examples of suitable resins include a polyhedral oligomeric silsesquioxane-based resin, a non-polyhedral oligomeric silsesquioxane epoxy resin, a poly(ethylene glycol) resin, a polyether resin (e.g., ring opened epoxies), an acrylic resin, an acrylate resin, a methacrylate resin, an amorphous fluoropolymer resin (e.g., CYTOP® from Bellex), and combinations thereof.

As used herein, the term “polyhedral oligomeric silsesquioxane” (commercially available under the tradename FOSS® from Hybrid Platics) refers to a chemical composition that is a hybrid intermediate (e.g., RSiO_{1.5}) between that of silica (SiO₂) and silicone (R₂SiO). An example of polyhedral oligomeric silsesquioxane can be that described in Kehagias et al., Microelectronic Engineering 86 (2009), pp. 776-778, which is incorporated by reference in its entirety. In an example, the composition is an organosilicon compound with the chemical formula [RSiO_{3/2}]ₙ, where the R groups can be the same or different. Example R groups for polyhedral oligomeric silsesquioxane include epoxy, azide/azido, a thiol, a poly(ethylene glycol), a norbornene, a tetrazine, acrylates, and/or methacrylates, or further, for example, alkyl, aryl, alkoxy, and/or haloalkyl groups. The resin composition disclosed herein may comprise one or more different cage or core structures as monomeric units. The average cage content can be adjusted during the synthesis, and/or controlled by purification methods, and a distribution of cage sizes of the monomeric unit(s) may be used in the examples disclosed herein.

In an example, the substrates 34A, 34A′ or 34B, 34B′ or 34C, 34C′ (whether single or multi-layered) may be round and have a diameter ranging from about 2 mm to about 300 mm, or may be a rectangular sheet or panel having its largest dimension up to about 10 feet (˜3 meters). In an example, the substrate 34A, 34A′ or 34B, 34B′ or 34C, 34C′ is a wafer having a diameter ranging from about 200 mm to about 300 mm. Wafers may subsequently be diced to form an individual flow cell substrate. In another example, the substrate 34A, 34A′ or 34B, 34B′ or 34C, 34C′ is a die having a width ranging from about 0.1 mm to about 10 mm. While example dimensions have been provided, it is to be understood that a substrate 34A, 34A′ or 34B, 34B′ or 34C, 34C′ with any suitable dimensions may be used. For another example, a panel may be used that is a rectangular support, which has a greater surface area than a 300 mm round wafer. Panels may subsequently be diced to form individual dies for the flow cells 30.

The flow cell 30 also includes the flow channel 26. While several flow channels 26 are shown in FIG. 2, it is to be understood that any number of flow channels 26 may be included in the flow cell 30 (e.g., a single channel 26, four channels 26, etc.). Each flow channel 26 may be isolated from each other flow channel 26 in the flow cell 30 so that fluid introduced into any particular flow channel 26 does not flow into any adjacent flow channel 26.

A portion of the flow channel 26 may be defined in the substrate 34A, 34A′ or 34B, 34B′ or 34C, 34C′ using any suitable technique that depends, in part, upon the material(s) of the substrate 34A, 34A′ or 34B, 34B′ or 34C, 34C′. In one example, a portion of the flow channel 26 is etched into a glass substrate, which is one example of the substrate 34A, 34A′. In this example, a lane 48, 48′ is defined in the substrate 34A, 34A′, and the space within the lanes 48, 48′ becomes part of the flow channel 26. In another example, a portion of the flow channel 26 may be patterned into a resin matrix material of a multi-layered structure using photolithography, nanoimprint lithography, etc. A separate material (e.g., material 44 in FIG. 3A, FIG. 3B, and FIG. 3C may be applied to the substrate 34A, 34A′ or 34B, 34B′ or 34C, 34C′ so that the separate material 44 defines at least a portion of the walls of the flow channel 26.

In an example, the flow channel 26 has a substantially rectangular configuration with rounded ends (as shown in FIG. 2). The length and width of the flow channel 26 may be smaller, respectively, than the length and width of the substrate 34A, 34A′ or 34B, 34B′ or 34C, 34C′ so that a portion of the substrate surface surrounding the flow channel 26 is available for attachment to another substrate 34A, 34A′ or 34B, 34B′ or 34C, 34C′ or to a lid, if desirable. In some instances, the width of each flow channel 26 can be at least about 1 mm, at least about 2.5 mm, at least about 5 mm, at least about 7 mm, at least about 10 mm, or more. In some instances, the length of each flow channel 26 can be at least about 10 mm, at least about 25 mm, at least about 50 mm, at least about 100 mm, or more. The width and/or length of each flow channel 26 can be greater than, less than or between the values specified above. In another example, the flow channel 26 is square (e.g., 10 mm×10 mm).

The depth of each flow channel 26 can be as small as a few monolayers thick, for example, when microcontact, aerosol, or inkjet printing is used to deposit the separate material 44 that defines the flow channel walls. In other examples, the depth of each flow channel 26 can be about 1 μm, about 10 μm, about 50 μm, about 100 μm, or more. In an example, the depth may range from about 10 μm to about 100 μm. In another example, the depth is about 5 μm or less. It is to be understood that the depth of each flow channel 26 can also be greater than, less than or between the values specified above. The depth of the flow channel 26 may also vary along the length and width of the flow cell 30, e.g., when posts 40, 40′ or depressions 42, 42′ are used.

In the example shown in FIG. 3A, each substrate 34A, 34A′ has a substantially flat surface 46, 46′; and the plurality of capture sites 32, 32′ are positioned in a pattern across the substantially flat surfaces 46, 46′.

The substantially flat surfaces 46, 46′ may be the bottom surface of the lanes 48, 48′ that are defined in the single layer substrate. While not shown, it is to be understood that a lane 48, 48′ may also be defined in the patterned layer 38, 38′ of a multi-layered substrate 34B, 34B′, 34C, 34C′. The lanes 48, 48′ may be etched into the substrate or defined, e.g., by lithography or another suitable technique.

The plurality of capture sites 32, 32′ is positioned in a pattern across the substantially flat surface 46, 46′.

Many different patterns for the capture sites 32, 32′ may be envisaged, including regular, repeating, and non-regular patterns. In an example, the capture sites 32, 32′ are disposed in a hexagonal grid for close packing and improved density. Other layouts may include, for example, rectangular layouts, triangular layouts, and so forth. In some examples, the layout or pattern can be an x-y format of capture sites 32, 32′ that are in rows and columns. In some other examples, the layout or pattern can be a repeating arrangement of capture sites 32, 32′ separated by regions of the substantially flat substrate 46, 46′. In still other examples, the layout or pattern can be a random arrangement of capture sites 32, 32′. The pattern may include stripes, swirls, lines, triangles, rectangles, circles, arcs, checks, diagonals, arrows, and/or squares.

The layout or pattern of the capture sites 32, 32′ may be characterized with respect to the density of the capture sites 32, 32′ (e.g., number of capture sites 32, 32′) in a defined area. For example, the capture sites 32, 32′ may be present at a density of approximately 2 million per mm². The density may be tuned to different densities including, for example, a density of about 100 per mm², about 1,000 per mm², about 0.1 million per mm², about 1 million per mm², about 2 million per mm², about 5 million per mm², about 10 million per mm², about million per mm², or more, or less. It is to be further understood that the density of capture sites 32, 32′ can be between one of the lower values and one of the upper values selected from the ranges above. As examples, a high density array may be characterized as having capture sites 32, 32′ separated by less than about 100 nm, a medium density array may be characterized as having capture sites 32, 32′ separated by about 400 nm to about 1 μm, and a low density array may be characterized as having capture sites 32, 32′ separated by greater than about 1 μm. While example densities have been provided, it is to be understood that any suitable densities may be used. In some instances, it may be desirable for the spacing between capture sites 32, 32′ to be even greater than the examples listed herein.

The layout or pattern of the capture sites 32, 32′ may also or alternatively be characterized in terms of the average pitch, or the spacing from the center of one capture site 32, 32′ to the center of an adjacent capture site 32, 32′ (center-to-center spacing) or from the left edge of one capture site 32, 32′ to the right edge of an adjacent capture site 32, 32′ (edge-to-edge spacing). The pattern can be regular, such that the coefficient of variation around the average pitch is small, or the pattern can be non-regular in which case the coefficient of variation can be relatively large. In either case, the average pitch can be, for example, about 50 nm, about 0.1 μm, about 0.5 μm, about 1 μm, about 5 μm, about 10 μm, about 100 μm, or more or less. The average pitch for a particular pattern of capture sites 32, 32′ can be between one of the lower values and one of the upper values selected from the ranges above. In an example, the capture sites 32, 32′ have a pitch (center-to-center spacing) of about 1.5 μm. While example average pitch values have been provided, it is to be understood that other average pitch values may be used.

The capture sites 32, 32′ may have any suitable shape, geometry and dimensions, which may depend, at least in part, on the sequencing nanoparticle 10B that is to be captured by the capture site 32, 32′.

The capture sites 32, 32′ may be chemical capture sites, electrostatic captures sites, or magnetic capture sites.

Chemical capture sites include any example of the chemical capture agent set forth herein that can be deposited on or otherwise attached to predefined locations of the substantially flat surface 46, 46′. In one example, the chemical capture agent may be deposited, e.g., using microcontact printing, aerosol printing, etc., in a desirable location on the substantially flat surface 46, 46′ to form the capture sites 32, 32′. In another example, a mask (e.g., a photoresist) may be used to define the space/location where the chemical capture agent will be deposited. The chemical capture agent may then be deposited, and the mask removed (e.g., via lift-off, dissolution, or another suitable technique). In this example, the chemical capture agent may form a monolayer or thin layer of the chemical capture agent. In still another example, a polymer grafted with capture nucleic acids may be selectively applied to the substantially flat surface 46, 46′ to form the chemical captures sites.

Electrostatic captures sites include any example of the electrostatic capture agents set forth herein that can be deposited on predefined locations of the substantially flat surface 46, 46′. For example, electrode materials may be deposited using chemical vapor deposition, masking and deposition, or another suitable technique to form the capture sites 32, 32′. When electrostatic capture sites are used, the substrate 34A, 34A′ may include additional circuitry to address the individual capture sites 32, 32′.

Magnetic capture sites include any example of the magnetic capture agent set forth herein that can be deposited on predefined locations of the substantially flat surface 46, 46′. For example, magnetic materials may be deposited using chemical vapor deposition, masking and deposition, or another suitable technique to form the capture sites 32, 32′.

In the example of FIG. 3A, areas of the substantially flat surface 46, 46′ that do not contain the capture sites 32, 32′ function as interstitial regions between the capture sites 32, 32′.

In the example shown in FIG. 3B, the substrate 34B, 34B′ includes posts 40, 40′ separated by interstitial regions 50, 50′; and a capture site 32, 32′ is positioned over each of the posts 40, 40′.

Each post 40, 40′ is a three-dimensional structure that extends outward (upward) from an adjacent surface. The post 40, 40′ is thus a convex region with respect to the interstitial regions 50, 50′ that surround the posts 40, 40′. Posts 40, 40′ may be formed in or on a substrate 34B, 34B′. In FIG. 3B, the posts 40, 40′ are formed in the substrate 34B, 34B′. When the post 40, 40′ is formed “in the substrate,” it is meant that the layer 38, 38′ is patterned (e.g., via etching, photolithography, imprinting, etc.,) so that the resulting posts 40, 40′ extend above the adjacent surrounding interstitial regions 50, 50′. Alternatively, when the post 40, 40′ is formed “on the substrate,” it is meant that an additional material may be deposited on the substrate (e.g., on the single layer substrate) so that it extends above the underlying substrate.

The layout or pattern of the posts 40, 40′ may be any of the examples set forth herein for the capture sites 32, 32′. The layout or pattern of the posts 40, 40′ may be characterized with respect to the density of the posts 40, 40′ (e.g., number of posts 40, 40′) in a defined area. Any of the densities set forth for the capture sites 32, 32′ may be used for the posts 40, 40′. The layout or pattern of the posts 40, 40′ may also be characterized in terms of the average pitch, or the spacing from the center of one post 40, 40′ to the center of an adjacent post 40, 40′ (center-to-center spacing) or from the left edge of one post 40, 40′ to the right edge of an adjacent post 40, 40′ (edge-to-edge spacing). Any of the average pitches set forth for the capture sites 32, 32′ may be used for the posts 40, 40′.

While any suitable three-dimensional geometry may be used for the posts 40, 40′, a geometry with an at least substantially flat top surface may be desirable so that the capture site 32, 32′ may be formed thereon. Example post geometries include a cylinder, a cube, polygonal prisms (e.g., rectangular prisms, hexagonal prisms, etc.), or the like.

The size of each post 40, 40′ may also be characterized by its top surface area, height, and/or diameter.

The top surface area of each post 40, 40′ can be selected based upon the size of the sequencing nanoparticle 10A, 10B that is to be anchored to the capture site 32, 32′ that is supported by the post 40, 40′. For example, the top surface area of each post 40, 40′ can be at least about 1×10⁻⁴ μm², at least about 1×10⁻³ μm², at least about 0.1 μm², at least about 1 μm², at least about 10 μm², at least about 100 μm², or more. Alternatively or additionally, the top surface area of each post 40, 40′ can be at most about 1×10⁴ μm², at most about 100 μm², at most about 10 μm², at most about 1 μm², at most about 0.1 μm², at most about 1×10⁻² μm², or less. The area occupied by each post top surface can be greater than, less than or between the values specified above.

The height of each post 40, 40′ can depend upon the channel 26 dimensions (if the flow cell 30 has a channel 26). In an example, the height may be at least about 0.1 μm, at least about 0.5 μm, at least about 1 μm, at least about 10 μm, at least about 100 μm, or more. Alternatively or additionally, the height can be at most about 1×10³ μm, at most about 100 μm, at most about 10 μm, or less. In some examples, the depth is about 0.4 μm. The height of each post 40, 40′ can be greater than, less than or between the values specified above.

In some instances, the diameter or each of the length and width of each post 40, 40′ can be at least about 50 nm, at least about 0.1 μm, at least about 0.5 μm, at least about 1 μm, at least about 10 μm, at least about 100 μm, or more. Alternatively or additionally, the diameter or each of the length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or each of the length and width is about 0.4 μm. The diameter or each of the length and width of each post 40, 40′ can be greater than, less than or between the values specified above.

In the example shown in FIG. 3B, a respective capture site 32, 32′ is positioned on each of the posts 40, 40′. The capture sites 32, 32′ may be chemical capture sites, electrostatic captures sites, or magnetic capture sites.

Chemical capture sites include any example of the chemical capture agent set forth herein that can be deposited on or otherwise attached to the top surface of each post 40, 40′. In one example, the chemical capture agent may be deposited, e.g., using microcontact printing, aerosol printing, etc., on each post 40, to form the capture site 32, 32′. In another example, a mask (e.g., a photoresist) may be used to cover the interstitial regions 50, 50′ and not the posts 40′. The chemical capture agent may then be deposited on the exposed posts 40′, and the mask removed (e.g., via lift-off, dissolution, or another suitable technique). In this example, the chemical capture agent may form a monolayer or thin layer of the chemical capture agent on the post 40, 40′. In still another example, a polymer grafted with capture nucleic acids may be selectively applied to the top surface of each post 40, 40′ to form the chemical captures sites.

Electrostatic captures sites include any example of the electrostatic capture agent set forth herein that can be deposited on the top surface of each post 40′. For example, electrode materials may be deposited using chemical vapor deposition, masking and deposition, or another suitable technique to form the capture sites 32, 32′. When electrostatic capture sites are used, the substrate 34B, 34B′ may include additional circuitry to address the individual capture sites 32, 32′.

Magnetic capture sites include any example of the magnetic capture agent set forth herein that can be deposited on the top surface of each post 40, 40′. For example, magnetic materials may be deposited using chemical vapor deposition, masking and deposition, or another suitable technique to form the capture sites 32, 32′.

In the example shown in FIG. 3C, the substrate 34C, 34C′ includes depressions 42, 42′ separated by interstitial regions 50, 50′; and a capture site 32, 32′ is positioned in each of the depressions 42, 42′.

Each depression 42, 42′ is a three-dimensional structure that extends inward (downward) from an adjacent surface. The depression 42, 42′ is thus a concave region with respect to the interstitial regions 50, 50′ that surround the depressions 42, 42′. Depressions 42, 42′ may be formed in a substrate 34C, 34C′. In the example shown in FIG. 3C, the layer 38, 38′ is patterned (e.g., via etching, photolithography, imprinting, etc.,) to define the depressions 42, 42′ so that the interstitial regions 50, 50′ extend above and surround the adjacent depressions 42, 42′.

The layout or pattern of the depressions 42, 42′ may be any of the examples set forth herein for the capture sites 32, 32′. The layout or pattern of the depressions 42, 42′ may be characterized with respect to the density of the depressions 42, 42′ (e.g., number of depressions 42, 42′) in a defined area. Any of the densities set forth for the capture sites 32, 32′ may be used for the depressions 42, 42′. The layout or pattern of the depressions 42, 42′ may also be characterized in terms of the average pitch, or the spacing from the center of one depression 42, 42′ to the center of an adjacent depression 42, 42′ (center-to-center spacing) or from the left edge of one depression 42, 42′ to the right edge of an adjacent depression 42, 42′ (edge-to-edge spacing). Any of the average pitches set forth for the capture sites 32, 32′ may be used for the depressions 42, 42′.

While any suitable three-dimensional geometry may be used for the depressions 42, 42′, a geometry with an at least substantially flat bottom surface may be desirable so that the capture site 32, 32′ may be formed thereon. Example depression geometries include a sphere, a cylinder, a cube, polygonal prisms (e.g., rectangular prisms, hexagonal prisms, etc.), or the like.

The size of each depression 42, 42′ may be characterized by its volume, opening area, depth, and/or diameter.

Each depression 42, 42′ can have any volume that is capable of receiving the material of the capture site 32, 32′. For example, the volume can be at least about 1×10⁻³ μm³, at least about 1×10⁻² μm³, at least about 0.1 μm³, at least about 1 μm³, at least about 10 μm³, at least about 100 μm³, or more. Alternatively or additionally, the volume can be at most about 1×10⁴ μm³, at most about 1×10³ μm³, at most about 100 μm³, at most about 10 μm³, at most about 1 μm³, at most about 0.1 μm³, or less.

The area occupied by each depression opening can be selected based on the size of the sequencing nanoparticle 10A, 10B to be anchored by the capture site 32, 32′. It may be desirable for the sequencing nanoparticle 10A, 10B to enter the depression 42, 42′, and thus the area occupied by the depression opening may be bigger than the size of the sequencing nanoparticle 10A, 10B. For example, the area for each depression opening can be at least about 1×10⁻³ μm², at least about 1×10⁻² μm², at least about 0.1 μm², at least about 1 μm², at least about 10 μm², at least about 100 μm², or more. Alternatively or additionally, the area can be at most about 1×10³ μm², at most about 100 μm², at most about 10 μm², at most about 1 μm², at most about 0.1 μm², at most about 1×10⁻² μm², or less. The area occupied by each depression opening can be greater than, less than or between the values specified above.

The depth of each depression 42, 42′ is large enough to house at least the capture site 32, 32′. In one example, the depression 42, 42′ may be filled with the capture site 32, 32′. In this example, the sequencing nanoparticle 10A, becomes anchored to the capture site 32, 32′ but does not enter the depression 42, 42′. In another example, the depression 42, 42′ may be partially filled with the capture site 32, 32′. In this example, the sequencing nanoparticle 10A, 10B at least partially enters the depression 42, 42′ and becomes anchored to the capture site 32, 32′ in the depression 42, 42′. In an example, the depth may be at least about 0.1 μm, at least about 0.5 μm, at least about 1 μm, at least about 10 μm, at least about 100 μm, or more. Alternatively or additionally, the depth can be at most about 1×10³ μm, at most about 100 μm, at most about 10 μm, or less. In some examples, the depth is about 0.4 μm. The depth of each depression 42, 42′ can be greater than, less than or between the values specified above.

In some instances, the diameter or each of the length and width of each depression 42, 42′ can be at least about 50 nm, at least about 0.1 μm, at least about 0.5 μm, at least about 1 μm, at least about 10 μm, at least about 100 μm, or more. Alternatively or additionally, the diameter or each of the length and width can be at most about 1×10³ μm, at most about 100 μm, at most about 10 μm, at most about 1 μm, at most about 0.5 μm, at most about 0.1 μm, or less (e.g., about 50 nm). In some examples, the diameter or each of the length and width is about 0.4 μm. The diameter or each of the length and width of each depression 42, 42′ can be greater than, less than or between the values specified above.

In the example shown in FIG. 3C, the capture site 32, 32′ is positioned in each of the depressions 42, 42′. The capture sites 32, 32′ may be chemical capture sites, electrostatic captures sites, or magnetic capture sites

Chemical capture sites include any example of the chemical capture agent set forth herein that can be deposited on or otherwise attached to the bottom surface of each depression 42, 42′. In one example, the chemical capture agent may be deposited, e.g., using microcontact printing, aerosol printing, etc., on each depression 42, 42′ to form the capture sites 32, 32′. In another example, a mask (e.g., a photoresist) may be used to cover the interstitial regions 50, 50′ and not the depressions 42, 42′. The chemical capture agent may then be deposited in the exposed depression 42, 42′, and the mask removed (e.g., via lift-off, dissolution, or another suitable technique). In this example, the chemical capture agent may form a monolayer or thin layer of the chemical capture agent in the depression 42, 42′. In still another example, a polymer grafted with capture nucleic acids may be selectively applied to the bottom surface of each depression 42, 42′.

Electrostatic captures sites include any example of the electrostatic capture agent set forth herein that can be deposited on the bottom surface of each depression 42, 42′. For example, electrode materials may be deposited using chemical vapor deposition, masking and deposition, or another suitable technique to form the capture sites 32, 32′. When electrostatic capture sites are used, the substrate 34C, 34C′ may include additional circuitry to address the individual capture sites 32, 32′.

Magnetic capture sites include any example of the magnetic capture agent set forth herein that can be deposited on the bottom surface of each depression 42, 42′. For example, magnetic materials may be deposited using chemical vapor deposition, masking and deposition, or another suitable technique to form the capture sites 32, 32′.

While the example architectures shown in FIG. 3A, FIG. 3B, and FIG. 3C depict the sequencing nanoparticle 10A or 10B anchored at the captures sites 32, 32′, it is to be understood that the flow cell 30 does not include the sequencing nanoparticle 10A, 10B until they are introduced thereto, e.g., during sequencing.

Kits Including the Functionalized Plasmonic Nanostructures

Any example of the flow cell 30 and the sequencing nanoparticles 10B may be part of a kit. An example of the kit includes the flow cell 30 including a plurality of capture sites 32, 32′ and a suspension including a liquid carrier and a plurality of the sequencing nanoparticles 10A, 10B dispersed throughout the liquid carrier. Any example of the sequencing nanoparticles 10A, and any liquid carrier that does not solubilize the sequencing nanoparticles 10B may be included in the suspension. In the kit, the mechanism of the sequencing nanoparticles 10A, 10B is selected to be able to anchor the sequencing nanoparticles 10A, 10B to the capture site 32, 32′ of the flow cell 30 in the kit.

Sequencing Method

When the sequencing nanoparticles 10A, 10B are to be used in sequencing, they may first be used for the generation of template nucleic acid strands that are to be sequenced. This example method involves off-flow cell library template formation, hybridization, and amplification.

At the outset of template strand formation, library templates may be prepared from any nucleic acid sample (e.g., a DNA sample or an RNA sample). The DNA nucleic acid sample may be fragmented into single-stranded, similarly sized (e.g., <1000 bp) DNA fragments. The RNA nucleic acid sample may be used to synthesize complementary DNA (cDNA), and the cDNA may be fragmented into single-stranded, similarly sized (e.g., <1000 bp) cDNA fragments. During preparation, adapters may be added to the ends of any of the fragments. Through reduced cycle amplification, different motifs may be introduced in the adapters, such as sequencing primer binding sites, indices, and regions that are complementary to the primers 18, 20 on the sequencing nanoparticles 10A, 10B. In some examples, the fragments from a single nucleic acid sample have the same adapters added thereto. The final library templates include the DNA or cDNA fragment and adapters at both ends. The DNA or cDNA fragment represents the portion of the final library template that is to be sequenced.

A plurality of library templates may be introduced to a suspension that includes the liquid carrier and the sequencing nanoparticles 10A, 10B disclosed herein. Multiple library templates are hybridized, for example, to one of two types of primers 18, 20 immobilized at the surface of the sequencing nanoparticles 10A, 10B.

Amplification of the template nucleic acid strand(s) on the sequencing nanoparticles 10A, 10B may be initiated to form a cluster of the template strands at the surface of the sequencing nanoparticles 10A, 10B. In one example, amplification involves cluster generating. In one example of cluster generation, the library templates are copied from the hybridized primers by 3′ extension using a high-fidelity DNA polymerase. The original library templates are denatured, leaving the copies immobilized all around the sequencing nanoparticles 10A, 10B. Isothermal bridge amplification or some other form of amplification may be used to amplify the immobilized copies. For example, the copied templates loop over to hybridize to an adjacent, complementary primer, and a polymerase copies the copied templates to form double stranded bridges, which are denatured to form two single stranded strands. These two strands loop over and hybridize to adjacent, complementary primers and are extended again to form two new double stranded loops. The process is repeated on each template copy by cycles of isothermal denaturation and amplification to create dense clonal clusters on the sequencing nanoparticles 10A, 10B. Each cluster of double stranded bridges is denatured. In an example, the reverse strand is removed by specific base cleavage, leaving forward template strands. Clustering results in the formation of several template strands immobilized on the sequencing nanoparticles 10A, 10B. The sequencing nanoparticles 10A, 10B with the cluster of template strands immobilized at the surface are referred to herein as “sequence ready nanoparticles.” This example of clustering is referred to as bridge amplification, and is one example of the amplification that may be performed. It is to be understood that other amplification techniques may be used.

The sequence ready nanoparticles may be washed to remove unreacted library templates, etc. and suspended in a fresh carrier liquid.

The suspension, including the sequence ready nanoparticles, may then be introduced into the flow cell 30 including the plurality of capture sites 32, 32′, whereby at least some of the sequence ready nanoparticles respectively attach to at least some of the capture sites 32, 32′. As described herein, the sequencing nanoparticles 10A, 10B (and thus the sequence ready nanoparticles) include a functional agent, charged atoms, or a magnetic material that specifically binds, attaches, or is otherwise attracted (e.g., electrostatically, magnetically, etc.) to the capture site 32, 32′. The suspension may be allowed to incubate for a predetermined time to allow the sequence ready nanoparticles to become anchored. When electrostatic capture sites 32, 32′ are used, the individual sites 32, 32′ may be electrically addressed to move the sequence ready nanoparticles toward individual capture sites 32, 32′. In this example, the charged atoms at the surface of the sequence ready nanoparticles are attracted to the electrostatic capture sites 32, 32′ that are individually or globally addressed.

A wash cycle may be performed to remove any unanchored sequence ready nanoparticles.

Sequencing primers may then be introduced to the flow cell 30. The sequencing primers hybridize to a complementary portion of the sequence of the template strands of the sequence ready nanoparticles. These sequencing primers render the template strands ready for sequencing.

An incorporation mix including labeled nucleotides may then be introduced into the flow cell 30, e.g., via an input port. In addition to the labeled nucleotides, the incorporation mix may include water, a buffer, and polymerases capable of nucleotide incorporation. When the incorporation mix is introduced into the flow cell 30, the mix enters the flow channel 26 or flows across the open substrate, and contacts the anchored sequence ready nanoparticles.

The incorporation mix is allowed to incubate in or on the flow cell 30, and labeled nucleotides (including optical labels) are incorporated by respective polymerases into the nascent strands along the template strands on each of the sequence ready nanoparticles. During incorporation, one of the labeled nucleotides is incorporated, by a respective polymerase, into one nascent strand that extends one sequencing primer and that is complementary to one of the template strands. Incorporation is performed in a template strand dependent fashion, and thus detection of the order and type of labeled nucleotides added to the nascent strand can be used to determine the sequence of the template strand. Incorporation occurs in at least some of the template strands across the sequence ready nanoparticles during a single sequencing cycle.

The incorporated labeled nucleotides may include a reversible termination property due to the presence of a 3′ OH blocking group, which terminates further sequencing primer extension once the labeled nucleotide has been added. After a desired time for incubation and incorporation, the incorporation mix, including non-incorporated labeled nucleotides, may be removed from the flow cell 30 during a wash cycle. The wash cycle may involve a flow-through technique, where a washing solution (e.g., buffer) is directed into, through, and then out of flow channel 26, e.g., by a pump or other suitable mechanism. An open flow cell 30 may be sprayed, dunked, or otherwise exposed to the wash solution.

Without further incorporation taking place, the most recently incorporated labeled nucleotides can be detected through an imaging event. During the imaging event, an illumination system may provide an excitation light to the flow cell 30. The optical labels of the incorporated labeled nucleotides emit optical signals in response to the excitation light, and these optical signals can be imaged using a suitable imaging device.

After imaging is performed, a cleavage mix may then be introduced into or onto the flow cell 30. In an example, the cleavage mix is capable of i) removing the 3′ OH blocking group from the incorporated nucleotides, and ii) cleaving the optical label from the incorporated nucleotide. Examples of 3′ OH blocking groups and suitable de-blocking agents/components in the cleavage mix may include: ester moieties that can be removed by base hydrolysis; allyl-moieties that can be removed with NaI, chlorotrimethylsilane and Na₂S₂O₃ or with Hg(II) in acetone/water; azidomethyl which can be cleaved with phosphines, such as tris(2-carboxyethyl)phosphine (TCEP) or tri(hydroxypropyl)phosphine (THP); acetals, such as tert-butoxy-ethoxy which can be cleaved with acidic conditions; MOM (—CH₂OCH₃) moieties that can be cleaved with LiBF₄ and CH₃CN/H₂O; 2,4-dinitrobenzene sulfenyl which can be cleaved with nucleophiles such as thiophenol and thiosulfate; tetrahydrofuranyl ether which can be cleaved with Ag(I) or Hg(II); and 3′ phosphate which can be cleaved by phosphatase enzymes (e.g., polynucleotide kinase). Examples of suitable optical label cleaving agents/components in the cleavage mix may include: sodium periodate, which can cleave a vicinal diol; phosphines, such as tris(2-carboxyethyl)phosphine (TCEP) or tris(hydroxypropyl)phosphine (THP), which can cleave azidomethyl linkages; palladium and THP, which can cleave an allyl; bases, which can cleave ester moieties; or any other suitable cleaving agent.

Additional sequencing cycles may then be performed until the template strands are sequenced.

In other sequencing methods, the suspension of sequencing nanoparticles 10A, 10B may first be introduced into the flow cell 30 and exposed to conditions that help to anchor at least some of the sequencing nanoparticles 10A, 10B to the capture sites 32, 32. In these examples, the sequencing nanoparticles 10A, 10B do not have the cluster of template strands attached thereto. Rather, the library templates are prepared off-flow cell, and then are introduced into the flow cell 30 for hybridization and amplification of the template nucleic acid strands on the already anchored sequencing nanoparticles 10A, 10B. In this example, any unattached library templates are removed from the flow cell 30 prior to sequencing and then sequencing may then be performed as described herein.

To further illustrate the present disclosure, examples are given herein. It is to be understood that these examples are provided for illustrative purposes and are not to be construed as limiting the scope of the present disclosure.

### EXAMPLES

### Example 1

Poly(lactic acid) nanoparticles were prepared using flash nanoprecipitation. The polymer was dissolved in acetone, and the solution was added to water at a volume ratio of 1:2. The mixture was rapidly stirred and the solvent and non-solvent were allowed to evaporate. The result was a unimodal distribution of PLA nanoparticles having a Z-average diameter (nm) of 142.2 nm±45.08 nm (measured using Dynamic Light Scattering). The mean zeta potential (mV) of the PLA nanoparticle distribution was −18.0 mV±4.18 mV (measured using a ZetaSizer), indicating that the nanoparticles were negatively charged.

The PLA nanoparticles were mixed with poly(N-(5-azidoacetamidylpentyl)acrylamide-co-acrylamide (PAZAM) and the mixture was allowed to incubate to adsorb the PAZAM on the PLA nanoparticles. The Z-average diameter (nm) and the mean zeta potential (mV) for the distribution of the PAZAM coated PLA nanoparticles were measured as described.

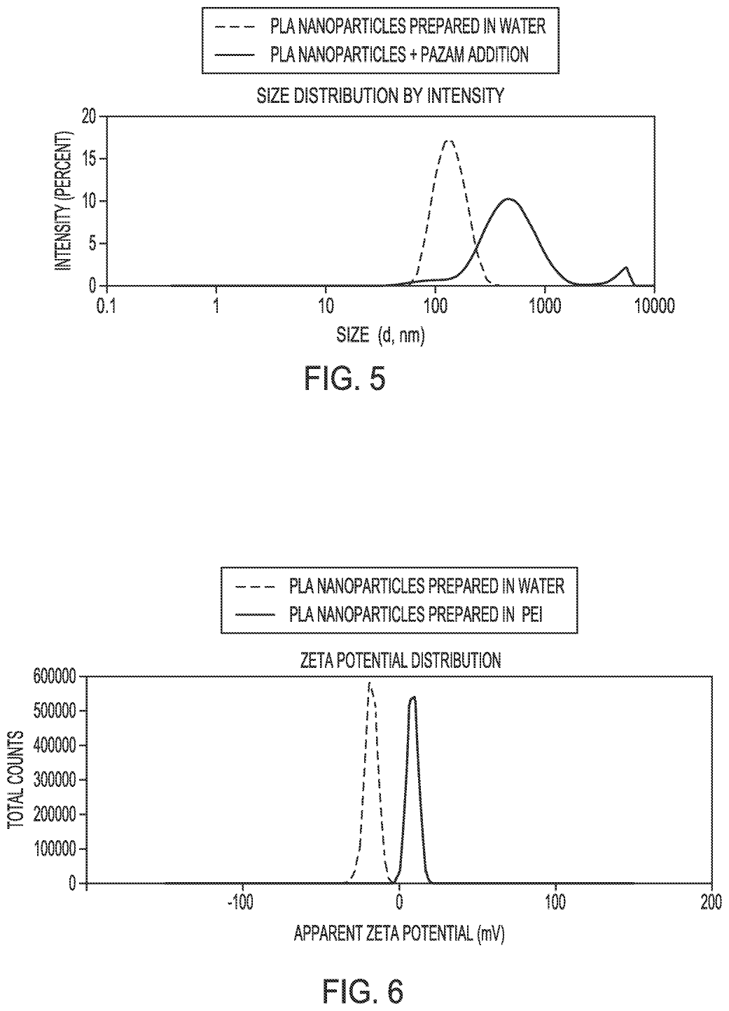

The zeta potential distribution for each of the PLA nanoparticles and the PAZAM coated PLA nanoparticles is shown in FIG. 4, which plots total counts (Y axis) versus the apparent zeta potential (mv). As noted above and as depicted in FIG. 4, the PLA nanoparticle distribution exhibited a negative charge. Also as depicted in FIG. 4, the PAZAM coated PLA nanoparticle distribution exhibited a positive charge (which may be due to quaternary ammonium cations residing on some of the PAZAM side chains). The size distribution for each of the PLA nanoparticles and the PAZAM coated PLA nanoparticles is shown in FIG. 5, which plots the intensity (%, Y axis) versus the size (diameter in nm, X axis). These results indicate a size increase when the PAZAM layer is added. It is to be understood that purification may be performed to improve the size and/or size distribution.

### Example 2

PAZAM with P5 and P7 primers pre-grafted thereto was used in this example. The mean zeta potential (mV) of this polymer was −34.6 mV±4.9 mV (measured using a ZetaSizer), indicating that the pre-grafted polymer was negatively charged.

Poly(lactic acid) nanoparticles were prepared with polyethyleneimine (PEI) at the surface. These particles were prepared using a modified flash nanoprecipitation method as described herein. The poly(lactic acid) was introduced into acetone and the mixture was agitated overnight to ensure dissolution of the poly(lactic acid). The PLA:acetone solution was added to polyethyleneimine at a volume ratio of 1:1. The mixture was rapidly stirred, which formed the positively charged PLA nanoparticles. The solvent was allowed to evaporate. The mean zeta potential (mV) for the distribution of the PEI coated PLA nanoparticles was measured as described. These results are plotted in FIG. 6 with the mean zeta potential results for the PLA nanoparticles from Example 1. As depicted, the PEI coated PLA nanoparticle distribution exhibited a positive charge.

Additional Notes

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

Reference throughout the specification to “one example,” “another example,” “an example,” and so forth, means that a particular element (e.g., feature, structure, and/or characteristic) described in connection with the example is included in at least one example described herein, and may or may not be present in other examples. In addition, it is to be understood that the described elements for any example may be combined in any suitable manner in the various examples unless the context clearly dictates otherwise.

It is to be understood that the ranges provided herein include the stated range and any value or sub-range within the stated range, as if such values or sub-ranges were explicitly recited. For example, a range from about 2 mm to about 300 mm, should be interpreted to include not only the explicitly recited limits of from about 2 mm to about 300 mm, but also to include individual values, such as about 40 mm, about 250.5 mm, etc., and sub-ranges, such as from about 25 mm to about 175 mm, etc.

Furthermore, when “about” and/or “substantially” are/is utilized to describe a value, they are meant to encompass minor variations (up to +/−10%) from the stated value.

While several examples have been described in detail, it is to be understood that the disclosed examples may be modified. Therefore, the foregoing description is to be considered non-limiting.

What is claimed is:

1. A method, comprising:
generating a negatively charged nanoparticle with a negatively chargeable, hydrophobic polymer;
in a layer-by-layer fashion, sequentially forming layers of a positively charged acrylamide hydrogel and of the negatively chargeable, hydrophobic polymer on the negatively charged nanoparticle to form a coated nanoparticle until i) a particle size of a dry form of the coated nanoparticle ranges from about 200 nm to about 1 μm, and ii) the positively charged acrylamide hydrogel forms an outer layer of the coated nanoparticle; and
grafting a negatively charged primer set to the outer layer.

2. The method as defined in claim 1, wherein the negatively chargeable, hydrophobic polymer is selected from the group consisting of poly(lactic acid) (PLA), poly(lactic-co-glycolic acid) (PLGA), poly(ε-caprolactone) (PCL), and poly(glycolic acid) (PGA).

3. The method as defined in claim 1, wherein generating the nanoparticle of the negatively chargeable, hydrophobic polymer involves flash nanoprecipitation.

4. The method as defined in claim 1, wherein the positively charged acrylamide hydrogel includes quaternary ammonium cations.

5. A method, comprising:
grafting a negatively charged primer set to an acrylamide hydrogel, thereby generating a negatively charged pre-grafted acrylamide hydrogel;
generating a nanoparticle having a core and a positively charged polymer coating at a surface of the core; and
attaching the negatively charged pre-grafted acrylamide hydrogel to the positively charged polymer coating.

6. The method as defined in claim 5, wherein generating the nanoparticle involves a modified flash nanoprecipitation process utilizing polyethyleneimine, chitosan, poly(L-lysine), or poly(diallyldimethylammonium chloride) poly(allylamine hydrochloride) (PAH) as a non-solvent phase during precipitation of the core.

7. The method as defined in claim 5, wherein the core includes a negatively chargeable, hydrophobic polymer selected from the group consisting of poly(lactic acid) (PLA), poly(lactic-co-glycolic acid) (PLGA), poly(ε-caprolactone) (PCL), and poly(glycolic acid) (PGA).

8. A sequencing nanoparticle, comprising:
a core of a negatively chargeable, hydrophobic polymer;
alternating layers of a positively charged acrylamide hydrogel and the negatively chargeable, hydrophobic polymer positioned on the core, wherein the positively charged acrylamide hydrogel forms an outer layer; and
a negatively charged primer set attached to the outer layer.

9. The sequencing nanoparticle as defined in claim 8, wherein:
the negatively chargeable, hydrophobic polymer is selected from the group consisting of poly(lactic acid) (PLA), poly(lactic-co-glycolic acid) (PLGA), poly(ε-caprolactone) (PCL), and poly(glycolic acid) (PGA); and
the positively charged acrylamide hydrogel includes quaternary ammonium cations.

10. A sequencing nanoparticle, comprising:
a core of a negatively chargeable, hydrophobic polymer;
a positively charged polymer coating attached to the core; and
a negatively charged pre-grafted acrylamide hydrogel attached to the positively charged polymer coating.

11. The sequencing nanoparticle as defined in claim 10, wherein:
the negatively chargeable, hydrophobic polymer is selected from the group consisting of poly(lactic acid) (PLA), poly(lactic-co-glycolic acid) (PLGA), poly(ε-caprolactone) (PCL), and poly(glycolic acid) (PGA);
the positively charged polymer coating is polyethyleneimine; and
the pre-grafted acrylamide hydrogel is poly(N-(5-azidoacetamidylpentyl)acrylamide-co-acrylamide having forward and reverse primers grafted thereto.
